(19) United States
(12) Patent Application Publication
(10) Pub. No.: US 2025/0276183 A1
(43) Pub. Date: Sep. 4, 2025

(54) CONTINUOUS UNIPOLAR IMPEDANCE MONITORING TO DIAGNOSE LEFT BUNDLE BRANCH AREA PACING LEAD STABILITY

(71) Applicant: Cardiac Pacemakers, Inc., St. Paul, MN (US)

(72) Inventor: Jessica Burr, Boston, MA (US)

(21) Appl. No.: 18/594,623

(22) Filed: Mar. 4, 2024

Related U.S. Application Data
(60) Provisional application No. 63/452,335, filed on Mar. 15, 2023.

(51) Int. Cl. A61N 1/362; A61N 1/02; A61N 1/05; A61N 1/37; A61N 1/375

(52) U.S. Cl. CPC A61N 1/3622; A61N 1/025; A61N 1/056; A61N 1/371; A61N 1/3756

(57) ABSTRACT

A method of operating a cardiac rhythm management (CRM) system includes recurrently calculating, by a medical device of the CRM system, impedances between a housing electrode included in a housing of the medical device and each of multiple pacing electrodes, wherein the multiple pacing electrodes include a left bundle branch pacing electrode configured for placement in a left bundle branch of a subject; comparing the calculated impedances to one or more specified impedance values; and producing an alert regarding placement of a pacing electrode in response to a calculated impedance corresponding to the pacing electrode differing from the one or more specified impedance values by a predetermined threshold impedance value.

### CLAIM OF PRIORITY

This application claims the benefit of U.S. Provisional Application No. 63/452,335 filed on Mar. 15, 2023, which is hereby incorporated by reference in its entirety.

### TECHNICAL FIELD

This document relates generally to cardiac rhythm management systems and particularly, but not by way of limitation, to methods, systems, and devices for automatic monitoring of cardiac pacing lead stability.

### BACKGROUND

The heart is the center of a person's circulatory system and includes an intrinsic electro-mechanical system for performing two major pumping functions.

The left portions of the heart, including a left atrium (LA) and a left ventricle (LV), draw oxygenated blood from the lungs and pump it to body organs to provide the organs with their metabolic need for oxygen. The right portions of the heart, including a right atrium (RA) and a right ventricle (RV), draw deoxygenated blood from the body organs and pump it to lungs where the blood gets oxygenated. These pumping functions result from contractions of the myocardium of the heart. In a normal heart, a sinoatrial (SA) node, the heart's natural pacemaker, generates intrinsic electrical pulses that propagate through an electrical conduction system to various regions of the heart to excite the myocardial tissues of the cardiac muscles. For example, intrinsic electrical pulses originating from the SA node propagate through an atrio-ventricular (AV) node that is between the RA and RV. From the AV node, a specialized intrinsic conduction system is used by the electrical impulses to reach ventricular myocardial tissues, resulting in contraction activities of ventricles. This specialized conduction system includes the His bundle, the right and left conduction bundle branches that extend along the septum between the RV and LV, and the purkinje fibers that contact the ventricular myocardial tissues.

Coordinated delays of the propagations of the intrinsic electrical pulses in a normal electrical conduction system cause the various portions of the heart to contract in synchrony which results in efficient pumping functions. Heart disease can alter the normal intrinsic conduction paths. A blocked or otherwise abnormal electrical conduction can cause the heart to contract dyssynchronously, resulting in poor hemodynamic performance that may diminish the amount of blood supplied to the heart and the rest of the body. For example, a block in conduction of the electrical pulses in either of the left bundle branch or the right bundle branch can cause dyssynchrony among the ventricles (RV and LV) of the heart. Blockage of the normal conduction paths can cause intrinsic electrical pulses to conduct along alternate pathways, which can cause one ventricle to contract later with respect to the other ventricle. In such events of cardiac malfunctioning, cardiac pacing therapy can be provided to resynchronize contractions of the ventricles of the heart.

### SUMMARY

Methods, systems, and devices to monitor the efficacy of pacing electrodes used for treatment of cardiac conduction disease are disclosed. Example 1 includes subject matter (such as a method of operating a medical device) comprising recurrently calculating, by the medical device, impedances between a housing electrode included in a housing of the medical device and each of multiple pacing electrodes, wherein the multiple pacing electrodes include a left bundle branch pacing electrode configured for placement in a left bundle branch of a subject; comparing the calculated impedances to one or more specified impedance values; and producing an alert regarding placement of a pacing electrode in response to a calculated impedance corresponding to the pacing electrode differing from the one or more specified impedance values by a predetermined threshold impedance value.

In Example 2, the subject matter of Example 1 optionally includes recurrently calculating the impedances between the housing electrode and each electrode of an implantable lead that includes the multiple pacing electrodes including the left bundle branch pacing electrode; and producing the alert regarding placement of the left bundle branch pacing electrode when the calculated impedance of the left bundle branch pacing electrode is greater than the one or more specified impedance values by the predetermined threshold impedance value.

In Example 3, the subject matter of Example 2 optionally includes recurrently calculating the impedances between the housing electrode and another lead electrode configured for bipolar pacing with the left bundle branch pacing electrode; and producing the alert regarding placement of the implantable lead when the calculated impedance of at least one of the left bundle branch pacing electrode and the other lead electrode is greater than the one or more specified impedance values by the predetermined threshold impedance value.

In Example 4, the subject matter of Example 3 optionally includes producing the alert when the calculated impedance between the housing electrode and the left bundle branch pacing electrode is less than the specified impedance by the predetermined threshold impedance value.

In Example 5, the subject matter of one or any combination of Examples 1-4 optionally includes the medical device activating the calculating of the impedances in response to the medical device determining presence of the left bundle branch pacing electrode.

In Example 6, the subject matter of Example 5 optionally includes the medical device activating the recurrent calculating of the impedances a specified duration of time after determining presence of the left bundle branch pacing electrode.

In Example 7, the subject matter of one or any combination of Examples 1-6 optionally includes initiating a capture threshold test in response to the calculated impedance differing from the specified impedance by more than the predetermined threshold impedance value; and producing the alert in response to a detected change in the capture threshold and the calculated impedance differing from the specified impedance by the predetermined threshold impedance value.

In Example 8, the subject matter of one or any combination of Examples 1-7 optionally includes initiating a sensing threshold test in response to the calculated impedance differing from the specified impedance by more than the predetermined threshold impedance value; and producing the alert in response to a detected change in the sensing threshold and the calculated impedance differing from the specified impedance by the predetermined threshold impedance value.

In Example 9, the subject matter of one or any combination of Examples 1-8 optionally includes measuring, by the medical device, a baseline impedance value between the housing electrode and the left bundle branch pacing electrode.

Example 10 includes subject matter (such as an apparatus) comprising a therapy circuit configured to provide electrical pacing energy to a left bundle branch of a subject when operatively connected to multiple pacing electrodes that include a left bundle branch pacing electrode; a housing to contain electronic circuits of the apparatus and including a housing electrode formed on the housing; a sensing circuit configured to sense at least one of voltage or current of each of the pacing electrodes relative to the housing electrode; and a control circuit operatively coupled to the therapy circuit and the sensing circuit. The control circuit includes an impedance measurement circuit configured to measure an impedance between the housing electrode and each of the pacing electrodes. The control circuit is configured to recurrently initiate an impedance measurement between the housing electrode and each of the multiple pacing electrodes, including the left bundle branch pacing electrode; compare the calculated impedances to one or more specified impedance values; and produce an alert regarding placement of a pacing electrode in response to the calculated impedance corresponding to the pacing electrode differing from the one or more specified impedances by a predetermined threshold impedance value.

In Example 11, the subject matter of Example 10 optionally includes a control circuit configured to recurrently initiate an impedance measurement between the housing electrode and each lead electrode of an implantable lead that includes the left bundle branch pacing electrode as a lead tip electrode; and produce an alert regarding placement of the implantable lead in response to the impedance measured for the left bundle branch pacing electrode being greater than the one or more specified impedance values by the predetermined threshold impedance value.

In Example 12, the subject matter of Example 11 optionally includes a control circuit configured to recurrently initiate an impedance measurement between the housing electrode and a ring electrode of the implantable lead configurable for bipolar pacing with the left bundle branch pacing electrode; and produce an alert regarding placement of the implantable lead in response to the impedance measured for at least one of the left bundle branch pacing electrode and the ring electrode being greater than the one or more specified impedance values by the predetermined threshold impedance value.

In Example 13, the subject matter of one or both of Examples 11 and 12 optionally includes a control circuit configured to produce the alert when the calculated impedance between the housing electrode and the left bundle branch pacing electrode is less than the specified impedance by the predetermined threshold impedance value.

In Example 14, the subject matter of one or any combinations of Examples 11-13 optionally includes a control circuit configured to determine presence of the pacing electrode configured for placement in the left bundle branch; and begin recurrently initiating the impedance measurement a specified time duration after determining the pacing electrode is present.

In Example 15, the subject matter of one or any combination of Examples 10-14 optionally includes a control circuit configured to initiate a capture threshold test in response to the calculated impedance differing from the specified impedance by more than the predetermined threshold impedance value; and produce the alert in response to a detected change in the capture threshold and the calculated impedance differing from the specified impedance by the predetermined threshold impedance value.

In Example 16, the subject matter of one or any combination of Examples 10-15 optionally includes initiate a sensing threshold test in response to the calculated impedance differing from the specified impedance by more than the predetermined threshold impedance value; and produce the alert in response to a detected change in the sensing threshold and the calculated impedance differing from the specified impedance by the predetermined threshold impedance value.

In Example 17, the subject matter of one or any combination of Examples 10-16 optionally includes a control circuit configured to initiate a baseline impedance measurement between the housing electrode and the left bundle branch pacing electrode; and use the measured baseline impedance value as the specified impedance value.

Example 18 includes subject matter (such as a cardiac rhythm management system) or can optionally be combined with one or any combination of Examples of 1-17 to include such subject matter, comprising an implantable lead configured for placement in a right ventricle of a subject, the implantable lead having multiple pacing electrodes including a left bundle branch pacing electrode configured for placement in a left bundle branch of the subject; and a medical device for coupling to the implantable lead. The medical device includes a housing to contain electronic circuits of the apparatus and including a housing electrode formed on the housing; a therapy circuit configured to provide electrical pacing energy to the left bundle branch pacing electrode; a sensing circuit configured to sense at least one of voltage or current of each of the pacing electrodes relative to the housing electrode; and a control circuit operatively coupled to the therapy circuit and the sensing circuit that includes an impedance measurement circuit. The impedance measurement circuit is configured to measure an impedance between the housing electrode and each of the pacing electrodes. The control circuit is configured to recurrently initiate an impedance measurement between the housing electrode and each of the pacing electrodes of the lead, including the left bundle branch pacing electrode; compare the calculated impedances to one or more specified impedance values; and produce an alert regarding placement of the implantable lead in response to the calculated impedance corresponding to the pacing electrode differing from the one or more specified impedances by a predetermined threshold impedance value.

In Example 19, the subject matter of Example 18 optionally includes an implantable lead including a ring electrode configured for pacing a right ventricle of the subject. The control circuit is configured to produce the alert regarding placement of the implantable lead when the calculated impedance of at least one of the left bundle branch pacing electrode and the ring electrode is greater than the one or more specified impedance values by the predetermined threshold impedance value.

In Example 20, the subject matter of claim 19 optionally includes a control circuit configured to produce the alert regarding placement of the implantable lead when the calculated impedance between the housing electrode and the left bundle branch pacing electrode is less than the specified impedance by the predetermined threshold impedance value.

This summary is intended to provide an overview of the subject matter of the present application. It is not intended to provide an exclusive or exhaustive explanation of the invention. The detailed description is included to provide further information about the subject matter of the present patent application.

### BRIEF DESCRIPTION OF THE DRAWINGS

FIG. 1 illustrates portions of an example of a Cardiac Rhythm Management (CRM) system.

FIG. 2 illustrates portions of an example of an implantable medical device (IMD) of a CRM system and portions of the environment in which the IMD operates.

FIG. 3 shows an implantable lead having a retractable helix to anchor the lead end.

FIG. 4 is a block diagram of an example of an IMD of a CRM system.

FIG. 5 is a flow diagram of an example of a method of operating an IMD.

FIGS. 6A-6B are illustrations of an example of a placement of a Left Bundle Branch Area Pacing (LBBAP) electrode and an electrogram sensed using the electrode.

FIGS. 7A-7B are illustrations of another example of a placement of an LBBAP electrode and an electrogram sensed using the electrode.

FIGS. 8A-8B are illustrations of a further example of a placement of an LBBAP electrode and an electrogram sensed using the electrode.

FIG. 9 is a flow diagram of another example of a method of operating an IMD.

### DETAILED DESCRIPTION

Conventional right ventricular (RV) pacing therapy provides pacing pulses to the RV such as to provide relief to a subject suffering from blockage of normal conduction pathways of the right ventricle. Conduction system pacing (CSP) is the direct pacing of the conduction system of the heart, leading to a more physiological activation of the ventricles alternative to traditional right ventricular pacing. CSP therapy allows pacing at multiple positions within the conduction system (e.g., the His Bundle and the Left Bundle Branch) and multiple types of capture can result from pacing at these positions.

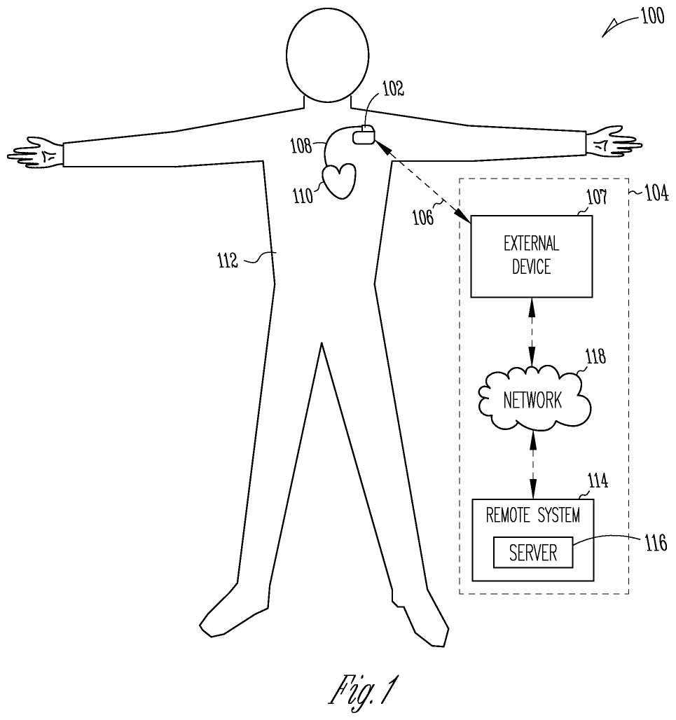

FIG. 1 illustrates portions of an example of a CRM system 100 and portions of an environment in which the CRM system 100 can be used. The CRM system 100 can be configured to include an implantable medical device (IMD) 102 and an external system 104, and a communication link such as a telemetry link 106. The IMD 102 can include an electronic unit coupled by a cardiac lead 108, or additional leads, to a heart 110 of a subject 112. Examples of the IMD 102 can include, but are not limited to, pacemakers, pacemaker/defibrillators, cardiac resynchronization devices, cardiac remodeling control devices, and cardiac monitors. In an example, the IMD 102 can be configured to monitor health of the heart 110 and determine one or more abnormalities associated with the heart 110. The IMD 102 can take a necessary action, such as stimulating one or more portions of the heart 110 through the lead 108, to treat the one or more abnormalities.

In an example, the external system 104 can include an external device 107 configured to communicate bi-directionally with the IMD 102 such as through the telemetry link 106. For example, the external device 107 can include a programmer to program the IMD 102 to provide one or more therapies to the heart 110. In an example, the external device 107 can program the IMD 102 to detect presence of a conduction block in a left bundle branch (LBB) of the heart 110 and prevent dyssynchronous contraction of the heart 110 by providing a cardiac resynchronization therapy (CRT) to the heart 110.

In an example, the external device 107 can be configured to transmit data to the IMD 102 through the telemetry link 106. Examples of such transmitted data can include programming instructions for the IMD 102 to acquire physiological data, perform at least one self-diagnostic test (such as for a device operational status), or deliver at least one therapy or any other data. In an example, the IMD 102 can be configured to transmit data to the external device 107 through the telemetry link 106. This transmitted data can include real-time physiological data acquired by the IMD 102 or stored in the IMD 102, therapy history data, an operational status of the IMD 102 (e.g., battery status or lead impedance), and the like. The telemetry link 106 can include an inductive telemetry link or a far-field radio-frequency telemetry link.

In an example, the external device 107 can be a part of a patient management system that can include other devices such as a remote system 114 for remotely programming the IMD 102. In an example, the remote system 114 can be configured to include a server 116 that can communicate with the external device 107 through a telecommunication network 118 such as to access the IMD 102 to remotely monitor the health of the heart 110 or adjust parameters associated with the one or more therapies.

FIG. 2 illustrates portions of another example of an IMD 102 of a CRM system and portions of the environment in which the IMD 102 operates. The IMD 102 can include a hermetically-sealed housing 204 and a header 206 that extends from the housing 204. The header 206 can include one or more receptacles such as for receiving proximal ends of one or more cardiac leads such as a lead 108A, a lead 108B and a lead 108C (collectively referred to herein as leads 108). Distal ends of the leads 108 can be coupled to electrodes such as for providing pacing energy, defibrillation energy, or both, in conjunction with the electrodes disposed on or around the heart 110. The electrodes can also be used for sensing electrical activity of the heart 110, including electrical activity related to contractions of the atria or ventricles.

As shown in FIG. 2, the heart 110 includes a right atrium (RA) 208, a left atrium (LA) 210, a right ventricle (RV) 212, a left ventricle (LV) 214, and a coronary sinus 216 extending from the RA 208. In an example, lead 108A can be an intravascular RA lead that can extend from a superior vena cava (SVC) into the RA 208 and can include electrodes such as a ring electrode 218 and a tip electrode 220 such as for sensing signals, or delivering pacing therapy, or both, to the RA 208.

In an example, lead 108C can be an intravascular right ventricle (RV) lead that can extend from the SVC into the RA 208, and then into the RV 212. The lead 108C can be configured to include a defibrillation coil electrode 226 such as to provide high energy shock therapy to the subject. The RV lead 108C can include an electrode pair 232 for sensing signals, delivering pacing therapy, or both. The RV lead 108C can be configured to achieve resynchronization of the RV 212.

In an example, lead 108B can be a Left Bundle Branch (LBB) area lead that can extend into the RA 208, into the RV 212, and then into the LBB area 230. In an example, the LBB area lead 108B can include tip electrode 222 and ring electrode 224 that can be used to deliver electro-stimulation energy and to sense intrinsic electrical heart signals.

The IMD 102 can include a housing electrode 211 formed on the housing 204 of the IMD 102. The IMD 102 can provide unipolar pacing using any pacing electrode of the leads and the housing electrode 211, or provide bipolar pacing using an electrode pair. The IMD 102 can use unipolar sensing to sense intrinsic electrical cardiac signals using the housing electrode 211 and a lead electrode or use bipolar sensing to sense intrinsic electrical signals using bipolar electrode pairs.

To provide therapy for conduction disease of the LV 214, electrical stimulation pulses can be provided to a stimulation location in the LBB area 230. Left Bundle Branch Area Pacing (LBBAP) involves the placement of LBBAP lead 108B into the LBB or surrounding tissue on the left side of the interventricular septum. Bipolar LBBAP can be delivered using tip electrode 222 as the pacing cathode and ring electrode 224 as the pacing anode. Unipolar pacing can be delivered tip electrode 222 as the pacing cathode and housing electrode 211 as the pacing anode. This LBB pacing area 230 can range from two to ten times deeper than the traditional right ventricular lead depth, depending on the thickness of the septum.

With the increased septal depth of an LBBAP lead in comparison to RV pacing leads, there is a focus on impedances during the implant procedure and in follow-up monitoring during the life of the device. Measuring the impedance seen at an electrode can provide information of placement of the LBBAP electrodes. For example, because it is desired to place the LBBAP tip electrode 222 between the left side of the interventricular septum and the endocardium of the LV 214, there is a possibility of perforation into the cavity of the LV 214. As the LBBAP lead 108B is implanted, the impedance seen at the tip electrode 222 climbs as the septal depth of the electrode 222 increases. A sudden drop in impedance (e.g., a drop of more than 20% within a predetermined timeframe) during the positioning of the LBBAP lead 108B can be indicative of perforation into the LV cavity, which then requires the lead to be repositioned.

Unipolar impedance or bipolar impedance can be measured using the IMD 102. Unipolar impedance for tip electrode 222 can be measured by applying a known electric current between tip electrode 222 and housing electrode 211. Voltage resulting from the applied current can be measured and the impedance from the tip electrode 222 to the housing electrode 211 may then be calculated using Ohms Law. Unipolar impedance for any electrode of the CRM system can be measured similarly. Bipolar impedance is measured by applying a known current between the tip electrode 222 (typically the pacing cathode) and ring electrode 224 (typically the pacing anode), and measuring the resulting voltage between the tip electrode 2222 and the ring electrode 224.

Monitoring unipolar impedance of the tip electrode 222 instead of bipolar impedance during implant accurately tracks the positioning of the pacing cathode without the advancing of the pacing anode causing confusion, which may happen for example if the pacing anode (e.g., ring electrode 224) is not full exposed from a delivery catheter or if the anode is embedded within the septum.

Post implant of the lead, the impedance of one or more electrodes of the lead remains a key indicator of the stability of the lead placement. A dramatic decrease in unipolar impedance of the tip electrode 222 that occurs after implant while the patient is mobile may indicate a positional change in the pacing lead 108B such as movement of the tip electrode 222 into the LV cavity. A dramatic increase in impedance can also provide information.

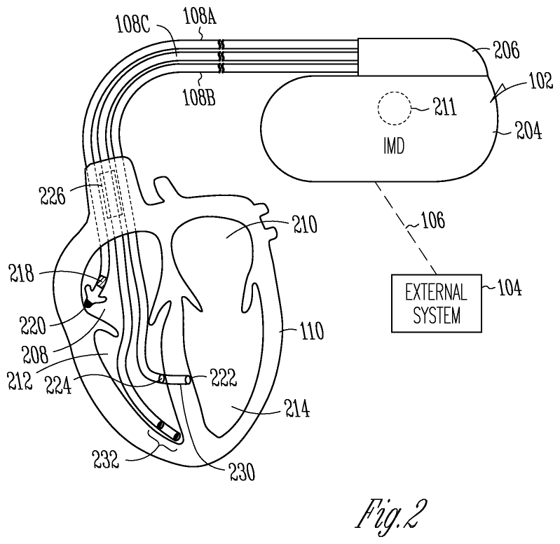

FIG. 3 shows an implantable lead 308 having a retractable helix 340 to anchor the lead end. The helix can be part of the tip electrode of a pacing lead. The helix 340 is retracted during initial positioning to allow for easier lead insertion, and the helix 340 is exposed when the lead is positioned to anchor the lead in the LBB. The top of FIG. 3 shows the helix 340 extended in the original implanted position. The bottom of FIG. 3 shows the helix 340 has later become retracted. A retracted helix or partially retracted helix may cause the unipolar impedance of the electrode to sharply increase. This may require redeployment of the helix 340.

Returning to FIG. 2, a change in position of the pacing anode may also be detected using unipolar impedance. For example, a sudden increase in impedance of the ring electrode 224 may indicate that the ring electrode 224 has moved from inside the RV cavity to inside the septum. If the ring electrode 224 was positioned in the septum at implant, a sudden decrease in impedance may indicate that the electrode 224 has moved outside the septum. A sudden change in unipolar impedance of any of the electrodes in FIG. 2 can provide information about the stability of the lead that includes the electrodes.

Therefore, monitoring the electrode impedance beyond the implant stage and outside of a clinical setting can provide information on lead stability throughout the life of the implanted CRM system. The impedance measured is the unipolar impedance regardless of the programmed pacing configuration. This means that the unipolar impedance is monitored despite the electrode being programmed to a bipolar configuration. Thus, the measured impedances are not necessarily reflective of the programmed electrode configuration of the CRM system.

FIG. 4 is a block diagram of portions of an example of an IMD 102 of a CRM system (e.g., CRM system 100 of FIG. 1) that can monitor the impedances of the electrodes of the CRM system to monitor stability of the leads of the CRM system. The IMD 102 can include a cardiac signal sensing circuit 404, a therapy circuit 406, and a control circuit 408. The IMD 102 can be connected to one or more implantable cardiac leads that include implantable electrodes. The IMD 102 includes a housing to contain electronic circuits of the IMD 102 and the housing includes at least one housing electrode. In some examples, the IMD 102 is a leadless medical device and the implantable electrodes used for sensing and pacing are included on the housing of the IMD 102. The electrodes include at least one LBB electrode for placement in or near the LBB of a patient.

The therapy circuit 406 provides electrical pacing energy to the LBB of the patient when operatively connected to pacing electrodes of the system including an LBBAP electrode. The sensing circuit 404 includes one or more sense amplifiers to sense one or both of a voltage signal or a current signal at electrodes of the system including LBBAP electrodes (e.g., LBBAP electrodes 222 and 224 in FIG. 2). The control circuit 408 may include a digital signal processor, application specific integrated circuit (ASIC), field programmable gate array (FPGA), microprocessor, or other type of processor, interpreting or executing instructions in software or firmware. In some examples, the control circuit 408 may include a state machine or sequencer that is implemented in hardware circuits. The control circuit 408 may include any combination of hardware, firmware, or software. The control circuit 408 includes one or more circuits to perform the functions described herein. A circuit may include software, hardware, firmware or any combination thereof. For example, the circuit may include instructions in software executing on the control circuit 408. Multiple functions may be performed by one or more circuits. The control circuit 408 includes an impedance measurement circuit 410 to measure the impedance between the housing electrode and each of the pacing electrodes.

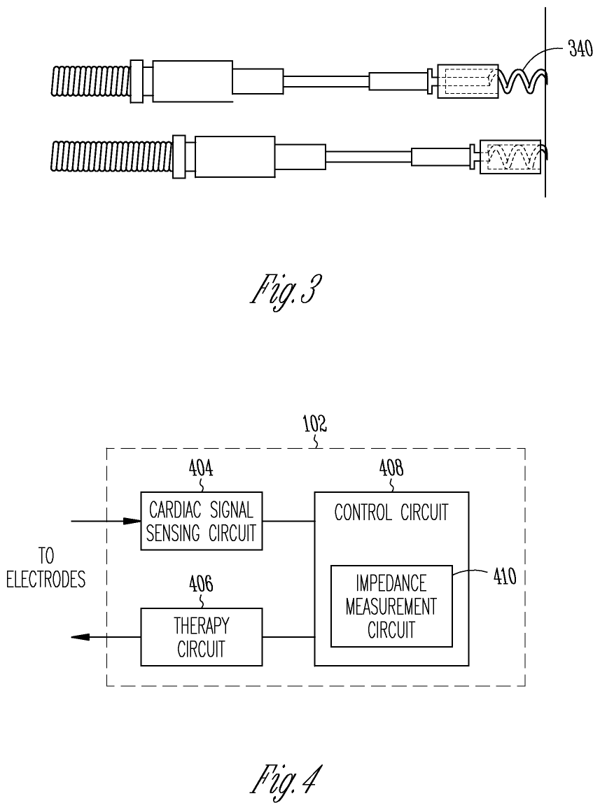

FIG. 5 is a flow diagram of an example of a method 500 of operating an implantable medical device (e.g., the IMD 102 of FIG. 4) of a CRM system to monitor the stability of the electrodes of the system throughout the lifetime of the system. To monitor the stability, the unipolar impedance of all implanted electrodes is monitored throughout the life of the device.

At 505, the control circuit 408 recurrently initiates an impedance measurement between the housing electrode and each of the pacing electrodes of the CRM system; including the unipolar impedance of the LBBAP electrodes. If the LBBAP electrodes are included in an implantable LBB lead, monitoring the unipolar impedance of the LBBAP electrodes gives additional insight into the lead stability. In some examples, the unipolar impedance measurement feature of the device can be enabled or disabled by a clinician (e.g., using external device 107 in FIG. 1). In some examples, the external device provides a prompt (e.g., on a display of the external device) to turn on the impedance measurement feature if the LBBAP electrode configuration is selected by the clinician. In some examples, the control circuit 408 is able to detect the presence of the LBBAP electrodes (e.g., by detecting presence of an LBB pacing lead). The control circuit 408 automatically enables the impedance measurements when the LBB pacing configuration is detected.

When enabled the unipolar impedance of the electrodes are measured using the impedance measurement circuit 410. A known current or voltage may be applied to the electrode and the resulting voltage or current may be sensed using the sensing circuit 404. The impedance circuit 410 calculates the impedance of the electrode using the current and voltage.

The impedance of all the electrodes may be measured, or only specified electrodes (e.g., only the LBBAP electrodes) can be measured. In some examples, the control circuit 408 does not initiate the unipolar impedance measurement right away when enabled. Instead, the control circuit waits a specified time duration after implant to begin the impedance measurements. This gives the patient time to become ambulatory with the CRM system, and lead stability can be assessed with activity of the patient. The time duration may be a programmed default time, or a time specified by the clinician. The time period between impedance measurements may also be a default time or a time specified by the clinician.

At block 510, the control circuit 408 compares a calculated impedance to a specified impedance value to see if the unipolar impedance of the electrode underwent a drastic change since the time of implant. In some examples, the impedances for the electrodes are determined at time of implant or soon after to allow for settling of the impedance as the tissue around the implant may scar. The measured values can be baseline values that are used as the specified impedance values. In some examples, the clinician inputs the values to the system at time of implant or near the beginning of life of the device. The clinician may specify one or both of a high impedance limit and a low impedance limit as the specified impedance values to which the calculated impedances will be compared. In some examples, the clinician specifies a delta impedance value and monitors for a change from the baseline impedance value by the delta impedance value.

At block 515, the control circuit 408 produces an alert regarding placement of an electrode in response to the calculated impedance for the electrode differing from the specified impedance by a predetermined threshold impedance value. The alert may be a flag that is displayed the next time the IMD 102 is interrogated by the external device. In some examples, the external device displays (e.g., using a user interface of the external device) the unipolar impedances of all the electrodes measured. The alert may include the external device changing the display of an impedance value (e.g., by highlighting the impedance value with a specific color) to bring the clinician's attention to the impedance value. In some examples, the alert is a signal sent to a patient device as a notification that there may be a problem and a lead or leads should be evaluated.

According to some examples, the control circuit 408 generates the alert in response to a sudden large increase in unipolar impedance of an LBBAP electrode that is greater than the predetermined threshold value. As explained previously herein, this may be due to retraction of the helix portion of an LBBAP cathode, retraction of the cathode into myocardial tissue, or the LBBAP anode (e.g., a ring electrode) entering the septal tissue. In some examples, the control circuit 408 generates the alert in response to a sudden large decrease in unipolar impedance of an LBBAP electrode that is less than the predetermined threshold value or is a decrease larger than the predetermined delta value. As explained previously herein, this may be due to the LBBAP cathode moving from the septum to the LV cavity or the LBBAP anode moving from the septum to the RV cavity. Monitoring multiple electrodes for impedance trends may improve the accuracy of the monitoring.

According to some examples, the unipolar the impedance measurement feature of the IMD 102 can be combined with other diagnostics. For examples, the IMD 102 may be able to monitor for changes in sensing thresholds of intrinsic cardiac signals or monitor for changes in pacing capture thresholds. A capture threshold refers to the threshold voltage needed to initiate a depolarization in the cardiac tissue. The IMD 102 may further check for changes in one or both of sensing and capture thresholds when detecting an impedance change for an electrode. This additional check can be useful to improve accuracy in the detecting whether a change in position of an electrode occurred. The control circuit 408 produces the alert when detecting the change in impedance and the detected change in the capture threshold or the sensing threshold. For example, the control circuit 408 would run an automatic threshold test and measure values of R-waves when detecting the sudden change in unipolar impedance. The control circuit 408 may generate the alert regarding lead stability when detecting an approximate 20% decrease in amplitude or magnitude of the R-wave and an approximate 20% increase in pacing threshold.

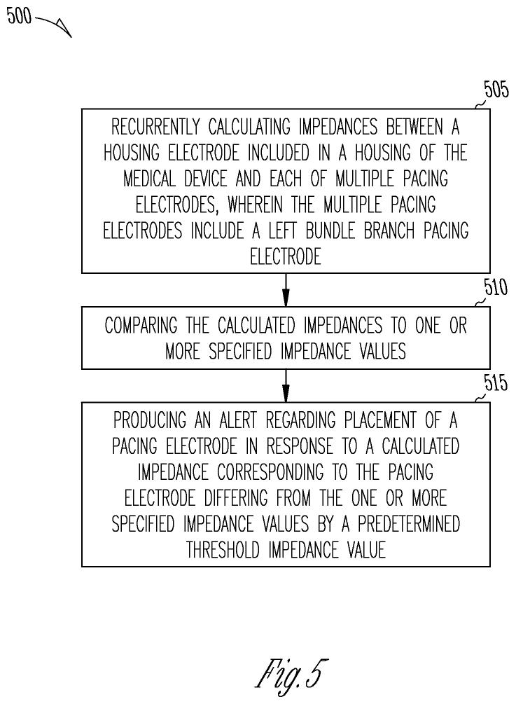

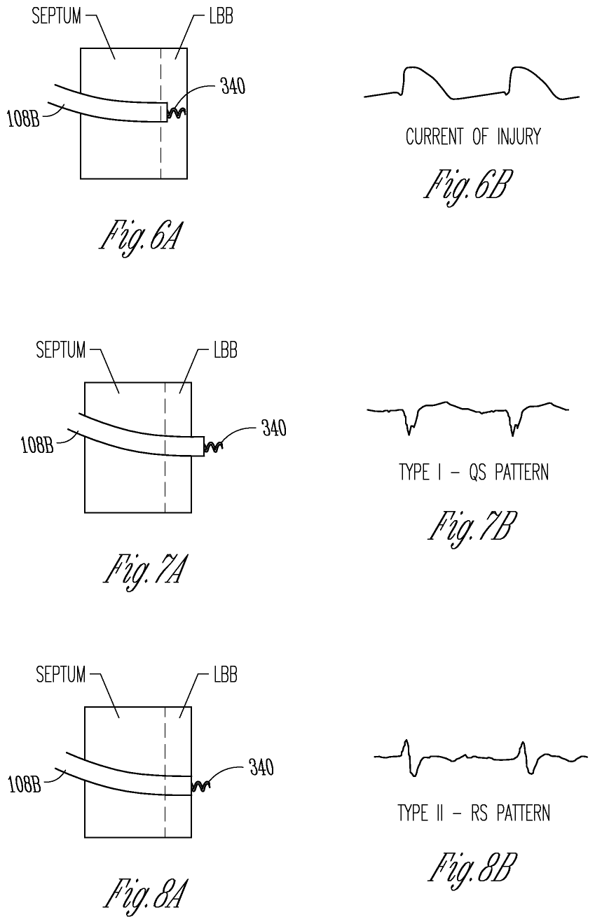

FIGS. 6A, 7A, and 8A illustrate placements of a LBBAP lead 108B in the LBB area. The dimensions in the Figures may be somewhat exaggerated for clarity of the illustrations. FIG. 6A illustrates placement of a tip electrode at the end of a LBBAP lead 108B (e.g., tip electrode 222 in FIG. 2) in the LBB. The electrode is positioned in the LBB portion of the septum. FIG. 7B is an example of an electrogram sensed by the sensing circuit 404 for the electrode position in FIG. 6A.

FIG. 7A illustrates an LBBAP electrode that perforated the endocardium of the LV. FIG. 7B is an example of an electrogram sensed by the sensing circuit 404 for the electrode position in FIG. 7A. The morphology of the signal changes and the magnitude of the R-wave decreases. The control circuit 408 may perform signal processing on the sensed electrogram to detect changes in one or both of the morphology and the R-wave magnitude of the sensed electrograms.

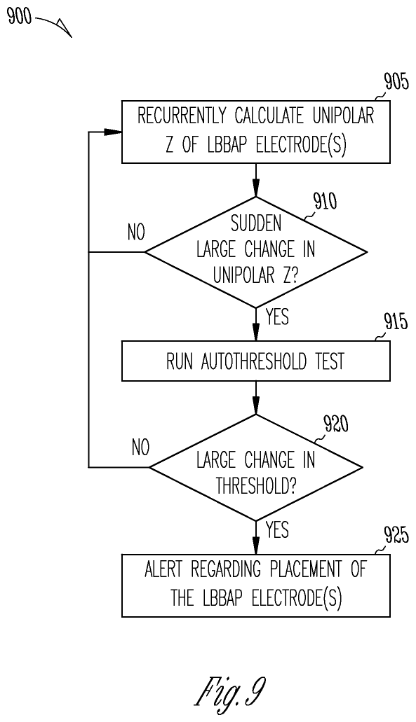

FIG. 8A illustrates an LBBAP electrode where just the helix perforated the endocardium of the LV, and the injured tissue is behind the helix. FIG. 8B is an example of an electrogram sensed by the sensing circuit 404 for the electrode position in FIG. 8A. The morphology of the signal changes and the magnitude of the R-wave decreases from the example of FIG. 6B. Detection of the changes in electrograms can be combined with detection of changes in unipolar impedance to detect a shift in position of the implanted lead. The alert generated by the IMD 102 calls attention to the change and a clinician can decide if the lead requires repositioning.

FIG. 9 is a flow diagram of an example of a method of operating an IMD of a CRM system to monitor stability of the LBBAP electrode or electrodes. At block 905, the unipolar impedance (unipolar Z) of one or both of an LBBAP cathode electrode and an LBBAP anode electrode are recurrently measured. In some examples, the measurements are made according to a schedule programmed into the IMD. The unipolar impedance of the LBBAP electrode or electrodes may be measured as part of a recurrent measurement of all electrodes of the CRM system.

At block 910, it is determined if there is a sudden change in the measured value unipolar impedance. As explained previously herein, the sudden change may be a sudden increase in impedance or a sudden decrease in impedance. The change in impedance may be “sudden” if the impedance value changed by more than a predetermined threshold impedance value within a specific time period. If a sudden change is not detected, the method returns to block 905 to wait for the next unipolar impedance measurement.

At block 915, if there is a sudden change in unipolar impedance detected, the IMD runs one or both of an automatic pacing threshold test and am automatic sensing threshold test. At block 920, if there is a significant change in either the pacing threshold or the sensing threshold, an alert regarding stability of the LBBAP electrode or electrodes is produced by the IMD. The alert may include on or more of a measured unipolar impedance, a measured pacing threshold, and a measured sensing threshold.

Improper positioning of the cardiac leads can lead to reduced effectiveness of the cardiac therapy provided to a patient. The present methods, systems, and devices can provide effective monitoring of implanted cardiac leads to detect whether the leads are stable or have moved and need to be repositioned.

### ADDITIONAL DESCRIPTION

The above detailed description includes references to the accompanying drawings, which form a part of the detailed description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. These computer-readable media can include, but are not limited to, hard disks, removable magnetic disks, removable optical disks (e.g., compact disks and digital video disks), magnetic cassettes, memory cards or sticks, random access memories (RAM's), read only memories (ROM's), and the like. In some examples, a carrier medium can carry code implementing the methods. The term “carrier medium” can be used to represent carrier waves on which code is transmitted.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A method comprising:
recurrently calculating, by a medical device, impedances between a housing electrode included in a housing of the medical device and each of multiple pacing electrodes, wherein the multiple pacing electrodes include a left bundle branch pacing electrode configured for placement in a left bundle branch of a subject;
comparing the calculated impedances to one or more specified impedance values; and
producing an alert regarding placement of a pacing electrode in response to a calculated impedance corresponding to the pacing electrode differing from the one or more specified impedance values by a predetermined threshold impedance value.

2. The method of claim 1, including:
wherein the recurrently calculating the impedances includes recurrently calculating the impedances between the housing electrode and each electrode of an implantable lead that includes the multiple pacing electrodes including the left bundle branch pacing electrode; and
wherein producing the alert includes producing the alert regarding placement of the left bundle branch pacing electrode when the calculated impedance of the left bundle branch pacing electrode is greater than the one or more specified impedance values by the predetermined threshold impedance value.

3. The method of claim 2, including:
wherein the recurrently calculating the impedances includes recurrently calculating the impedances between the housing electrode and another lead electrode configured for bipolar pacing with the left bundle branch pacing electrode; and
wherein producing the alert includes producing the alert regarding placement of the implantable lead when the calculated impedance of at least one of the left bundle branch pacing electrode and the other lead electrode is greater than the one or more specified impedance values by the predetermined threshold impedance value.

4. The method of claim 3, wherein the producing the alert includes producing the alert when the calculated impedance between the housing electrode and the left bundle branch pacing electrode is less than the specified impedance by the predetermined threshold impedance value.

5. The method of claim 1, including the medical device activating the calculating of the impedances in response to the medical device determining presence of the left bundle branch pacing electrode.

6. The method of claim 5, including the medical device activating the recurrent calculating of the impedances a specified duration of time after determining presence of the left bundle branch pacing electrode.

7. The method of claim 1, including:
initiating a capture threshold test in response to the calculated impedance differing from the specified impedance by more than the predetermined threshold impedance value; and
wherein the producing the alert regarding placement of the pacing electrode includes producing the alert in response to a detected change in the capture threshold and the calculated impedance differing from the specified impedance by the predetermined threshold impedance value.

8. The method of claim 1, including:
initiating a sensing threshold test in response to the calculated impedance differing from the specified impedance by more than the predetermined threshold impedance value; and
wherein the producing the alert regarding placement of the pacing electrode includes producing the alert in response to a detected change in the sensing threshold and the calculated impedance differing from the specified impedance by the predetermined threshold impedance value.

9. The method of claim 1, including:
measuring, by the medical device, a baseline impedance value between the housing electrode and the left bundle branch pacing electrode; and
using the measured baseline impedance value as the specified impedance value for the left bundle branch pacing electrode.

10. An apparatus comprising:
a therapy circuit configured to provide electrical pacing energy to a left bundle branch of a subject when operatively connected to multiple pacing electrodes including a left bundle branch pacing electrode;
a housing to contain electronic circuits of the apparatus and including a housing electrode formed on the housing;
a sensing circuit configured to sense at least one of voltage or current of each of the pacing electrodes relative to the housing electrode; and
a control circuit operatively coupled to the therapy circuit and the sensing circuit, and including an impedance measurement circuit configured to measure an impedance between the housing electrode and each of the pacing electrodes;
wherein the control circuit is configured to:
recurrently initiate an impedance measurement between the housing electrode and each of the multiple pacing electrodes, including the left bundle branch pacing electrode;
compare the calculated impedances to one or more specified impedance values; and
produce an alert regarding placement of a pacing electrode in response to the calculated impedance corresponding to the pacing electrode differing from the one or more specified impedances by a predetermined threshold impedance value.

11. The apparatus of claim 10, wherein the control circuit is configured to:
recurrently initiate an impedance measurement between the housing electrode and each lead electrode of an implantable lead that includes the left bundle branch pacing electrode as a lead tip electrode; and
produce an alert regarding placement of the implantable lead in response to the impedance measured for the left bundle branch pacing electrode being greater than the one or more specified impedance values by the predetermined threshold impedance value.

12. The apparatus of claim 11, wherein the control circuit is configured to:
recurrently initiate an impedance measurement between the housing electrode and a ring electrode of the implantable lead configurable for bipolar pacing with the left bundle branch pacing electrode; and
produce an alert regarding placement of the implantable lead in response to the impedance measured for at least one of the left bundle branch pacing electrode and the ring electrode being greater than the one or more specified impedance values by the predetermined threshold impedance value.

13. The apparatus of claim 11, wherein the control circuit is configured to produce the alert when the calculated impedance between the housing electrode and the left bundle branch pacing electrode is less than the specified impedance by the predetermined threshold impedance value.

14. The apparatus of claim 11, wherein the control circuit is configured to:
determine presence of the pacing electrode configured for placement in the left bundle branch; and
begin recurrently initiating the impedance measurement a specified time duration after determining the pacing electrode is present.

15. The apparatus of claim 10, wherein the control circuit is configured to:
initiate a capture threshold test in response to the calculated impedance differing from the specified impedance by more than the predetermined threshold impedance value; and
produce the alert in response to a detected change in the capture threshold and the calculated impedance differing from the specified impedance by the predetermined threshold impedance value.

16. The apparatus of claim 10, wherein the control circuit is configured to:
initiate a sensing threshold test in response to the calculated impedance differing from the specified impedance by more than the predetermined threshold impedance value; and
produce the alert in response to a detected change in the sensing threshold and the calculated impedance differing from the specified impedance by the predetermined threshold impedance value.

17. The apparatus of claim 10, wherein the control circuit is configured to:
initiate a baseline impedance measurement between the housing electrode and the left bundle branch pacing electrode; and
use the measured baseline impedance value as the specified impedance value.

18. A cardiac rhythm management system, the system comprising:
an implantable lead configured for placement in a right ventricle of a subject, the implantable lead having multiple pacing electrodes including a left bundle branch pacing electrode configured for placement in a left bundle branch of the subject;
a medical device for coupling to the implantable lead, the medical device including:
a housing to contain electronic circuits of the apparatus and including a housing electrode formed on the housing;
a therapy circuit configured to provide electrical pacing energy to the left bundle branch pacing electrode;
a sensing circuit configured to sense at least one of voltage or current of each of the pacing electrodes relative to the housing electrode; and
a control circuit operatively coupled to the therapy circuit and the sensing circuit, and including an impedance measurement circuit configured to measure an impedance between the housing electrode and each of the pacing electrodes;
wherein the control circuit is configured to:
recurrently initiate an impedance measurement between the housing electrode and each of the pacing electrodes of the lead, including the left bundle branch pacing electrode;
compare the calculated impedances to one or more specified impedance values; and
produce an alert regarding placement of the implantable lead in response to the calculated impedance corresponding to the pacing electrode differing from the one or more specified impedances by a predetermined threshold impedance value.

19. The system of claim 18,
wherein the implantable lead includes a ring electrode configured for pacing a right ventricle of the subject; and
wherein the control circuit is configured to produce the alert regarding placement of the implantable lead when the calculated impedance of at least one of the left bundle branch pacing electrode and the ring electrode is greater than the one or more specified impedance values by the predetermined threshold impedance value.

20. The system of claim 19, wherein the control circuit is configured to produce the alert regarding placement of the implantable lead when the calculated impedance between the housing electrode and the left bundle branch pacing electrode is less than the specified impedance by the predetermined threshold impedance value.
